(19) United States
(12) Patent Application Publication
(10) Pub. No.: US 2009/0181091 A1
(43) Pub. Date: Jul. 16, 2009

(54) PRODUCT AND PROCESS TO REGULATE A GENE NETWORK INVOLVED IN CONSTITUTIVE INFLAMMATION AND EARLY CANCER PROGRESSION

(76) Inventors: Abhijit G. Banerjee, Winnipeg (CA); Rajinder P. Bhullar, Winnipeg (CA)

Correspondence Address: ADE & COMPANY INC., 2157 Henderson Highway, WINNIPEG, MB, R2G1P9, CA

(21) Appl. No.: 12/199,153

(22) Filed: Aug. 27, 2008

Related U.S. Application Data
(60) Provisional application No. 60/968,169, filed on Aug. 27, 2007.

(51) Int. Cl. A61K 9/14; C07H 21/02; C12N 15/63; A61K 31/7052; A61P 35/00

(52) U.S. Cl. 424/484; 536/24.5; 435/320.1; 514/44

(57) ABSTRACT

A recombinant construct arranged for expression of a specific hairpin microRNA which disrupts crosstalk between the two inflammatory pathways is described for intervening in early cancer progression events. Accordingly, the construct can be used as a molecular therapeutic approach to prevent cancer of epithelial origin in mammals, which include but are by no means limited to cancers of the head and neck, lung, breast, ovarian, and prostate tissues.

### PRIOR APPLICATION INFORMATION

The instant application claims the benefit of U.S. Provisional Patent Application 60/968,169, filed Aug. 27, 2007.

### BACKGROUND OF THE INVENTION

Cancers of the mouth cavity are the 3rd most common malignancy of developing nations and 6th most common cancer in the developed world, resulting in several hundred thousands of deaths each year. The vast majority (˜90%) of these malignancies involve neoplastic lesions in the squamous epithelial compartment of the mouth cavity, lip, and pharynx. Like most cancers, oral squamous cell carcinomas (OSCC) results from a series of discrete, irreversible and sequential alterations in genes that control cell growth and differentiation, together with genetic aberrations promoting invasion and metastasis. Although risk factors for OSCC, such as alcohol and tobacco consumption, are well recognized, the molecular mechanisms responsible for this malignancy are still not fully understood. In this regard, our laboratory has initiated approaches to investigate transcriptome expression profiles in OPLs using Affymetrix U133 oligonucleotide arrays (Banerjee AG et al., Mol. Cancer Ther. (2005) 4(6):865-875). The differential gene expression profiles obtained allow us to develop true biomarkers of diagnostic and prognostic value, as well as help validate proteins whose expression or activity contribute to tumor progression. Such genomic and proteomic analysis is already providing essential information about molecules uniquely expressed in precancerous or cancerous lesions and providing knowledge about novel therapeutic targets and their molecular mechanisms. Targets in precancerous lesions are particularly attractive as it helps prevent malignancy associated morbidity that has not changed for Head and Neck region cancers in last two decades (<50%).

The superfamily of small (21 kDa) GTP binding proteins (small G proteins) comprises subfamilies: Ras, Rho, ADP ribosylation factors (ARFs), Rab, and Ran, which act as molecular switches to regulate numerous cellular responses. Members of the Rho family of GTPases, include RhoA, -B, and -C, Rac1 and -2, and Cdc42. Guanine nucleotide exchange factors (GEFs) activate Rho proteins by catalyzing the replacement of bound GDP with STP. The GTP-bound form of Rho proteins specifically interact with their effectors or targets and transmit signals to downstream molecules transcription factor NF κβ and enzyme cascade of p42/44MAP kinases respectively (Saito S et al., J. Biol. Chem. (2004) 279, (8):7169-7179, Niiya F et al., Oncogene (2006): 25827-837, Scoumanne and Chen, Cancer Research (2006):66 (12): 6271.). Both are known to contribute to inflammatory processes that propel progression of precancerous lesions. Rho proteins are inactivated through the hydrolysis of bound GTP to GDP by intrinsic GTPase activity, assisted by GTPase activating proteins (GAPs). The Rho family of GTPases, participates in regulation of the actin cytoskeleton and cell adhesion and are also involved in regulation of smooth muscle contraction, cell morphology, cell motility, neurite retraction, cytokinesis, and cell transformation (Hall, A. Science (1998) 279:509-514). Ect2, a transforming protein with sequence similarity to the dbl homology (DH) domain proteins, associates with a subset of the Rho family proteins: RhoA, Cdc42, and Rac1. Ect2 phosphorylation, which is required for its exchange activity, occurs during G2 and M phases. Human Ect2 is involved in the regulation of cytokinesis. The human ECT2 (Epithelial Cell Transforming Sequence 2) gene is located on the long arm of chromosome 3, at 3q26 (Takai S, et al., Genomics (1995) 27(1):220-222), a region of increased copy number and expression in a large number of cancers (Bitter M A, et al., Blood (1985) 66(6):1362-1370; Kim D H, et al., Int J Cancer. (1995) 60(6):812-819; Brzoska P M, et al., Cancer Res. (1995) 55(14):3055-3059; Balsara B R, et al., Cancer Res. (1997) 57(11):2116-2120; Heselmeyer K, et al., Genes Chromosomes Cancer (1997) 19(4):233-240; Sonoda G, et al., Genes Chromosomes Cancer. (1997) 20(4):320-8). Data available from the National Cancer Institute indicates that human ect2 is overexpressed in cancers of the ovary, uterus, parathyroid, testis, brain, and colon. We are the first group to show that ECT-2 gene is dysregulated early in the development of oral pre-malignant lesions and because of the phenotype imparted by the function of this gene may be responsible in oral cancer progression. The ect2 gene is conserved at the sequence and functional levels in mammals and insects. The pebble gene in Drosophila is the orthologue of mouse (G1293331) and human ect2, and is required for initiation of cytokinesis (Lehner C F, J. Cell Sci. (1992) 103:1021-1030; Prokopenko S N, et al., Genes Dev (1999) 13(17):2301-2314).

### SUMMARY OF THE INVENTION

According to a first aspect of the invention, there is provided a micro-RNA comprising a nucleotide sequence as set forth in SEQ ID No. 1.

According to a second aspect of the invention, there is provided a vector comprising a micro-RNA comprising a nucleotide sequence as set forth in SEQ ID No. 1 operably linked to a suitable promoter.

According to a third aspect of the invention, there is provided a pharmaceutical composition comprising the vector as described above and a nanoparticle delivery matrix.

According to a fourth aspect of the invention, there is provided an inhibitory nucleic acid molecule that corresponds to or is complementary to at least a fragment of an Ect2 nucleic acid molecule and that decreases Ect2 expression in a cell.

According to a fifth aspect of the invention, there is provided a method of preventing cancer in an individual having a precancerous lesion of epithelial origin comprising administering to said individual an effective amount of an inhibitory nucleic acid molecule that corresponds to or is complementary to at least a fragment of an Ect2 nucleic acid molecule and that decreases Ect2 expression in a cell.

### BRIEF DESCRIPTION OF THE DRAWINGS

FIG. 1 is a prior art diagram showing ETC2's role in G1 to S phase transition in cancer progression.

FIG. 2 shows the target sequence for the micro RNA.

FIG. 3 shows an example of the lentiviral vector design for micro-RNA based therapeutics.

FIG. 4 is a flowchart describing the steps in the oral drug delivery formulation preparation.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

Cancers of the mouth cavity are the 3rd most common malignancy of developing nations and 6th most common cancer in the developed world, resulting in several hundred thousands of deaths each year. The vast majority (˜90%) of these malignancies involve neoplastic lesions in the squamous epithelial compartment of the mouth cavity, lip, and pharynx. Like most cancers, oral squamous cell carcinomas (OSCC) result from a series of discrete, irreversible and sequential alterations in genes that control cell growth and differentiation, together with genetic aberrations promoting invasion and metastasis. Ect2, a transforming protein encoding a guanine nucleotide exchange factor that associates with a subset of the Rho family proteins: RhoA, Cdc42, and Rac1 and helps in signal transduction to and crosstalk amongst both the NFkB and MAP kinase driven inflammatory pathways. Ect2 phosphorylation is required for its exchange activity, and has been shown to occur in both G1 to S and G2 to M phases of cell cycle transition.

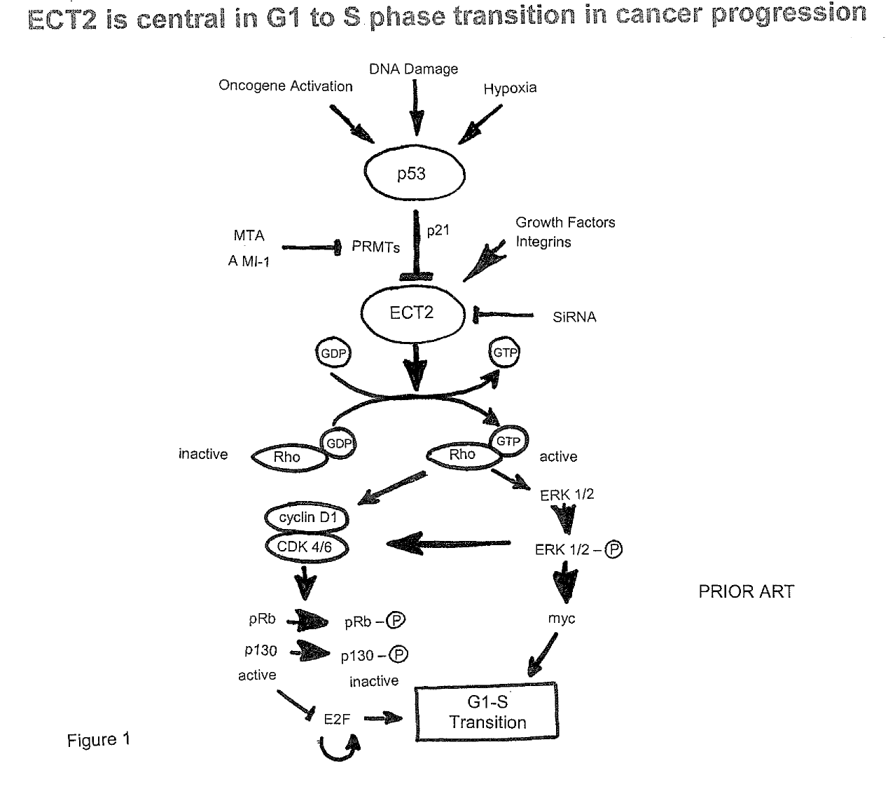

Described herein are a number of inhibitory nucleic acid molecule constructs for inhibiting expression of ect2 peptide. In one embodiment, there is a recombinant construct arranged for expression of a specific hairpin microRNA. In use, such a construct disrupts crosstalk between the two inflammatory pathways mentioned above and summarized in FIG. 1 to intervene in early cancer progression events. Accordingly, the construct can be used as a molecular therapeutic approach to prevent cancer of epithelial origin in mammals, which include but are by no means limited to cancers of the head and neck, lung, breast, ovarian, and prostate tissues. As will be appreciated by one of skill in the art, the treatment prevents cancer of epithelial origin in that the construct is administered to an individual who has at least one precancerous lesion in a tissue of epithelial origin, for example, in their mouth cavity and expression of the inhibitory nucleic acid molecule from the construct disrupts or intervenes in early cancer progression events. Accordingly, administration of an effective amount of a construct as described herein to an individual in need of such treatment, that is, an individual having at least one precancerous lesion in their mouth cavity will accomplish one or more of the following: prevent or slow progression of a precancerous lesion to oral cancer compared to an untreated control; and disrupt crosstalk between the NFkB and MAP kinase driven inflammatory pathways. An effective amount of such a microRNA or other inhibitory nucleic acid molecule may be determined by a variety of means and accordingly are with the average skill of one knowledgeable in the art and would not require undue experimentation. A suitable range however may be 5 to 500 mg/m²/day or 1 to 100 mg/kg, depending of course on many factors, not limited to the age, weight, general condition and severity of symptoms of the individual to be treated (patient).

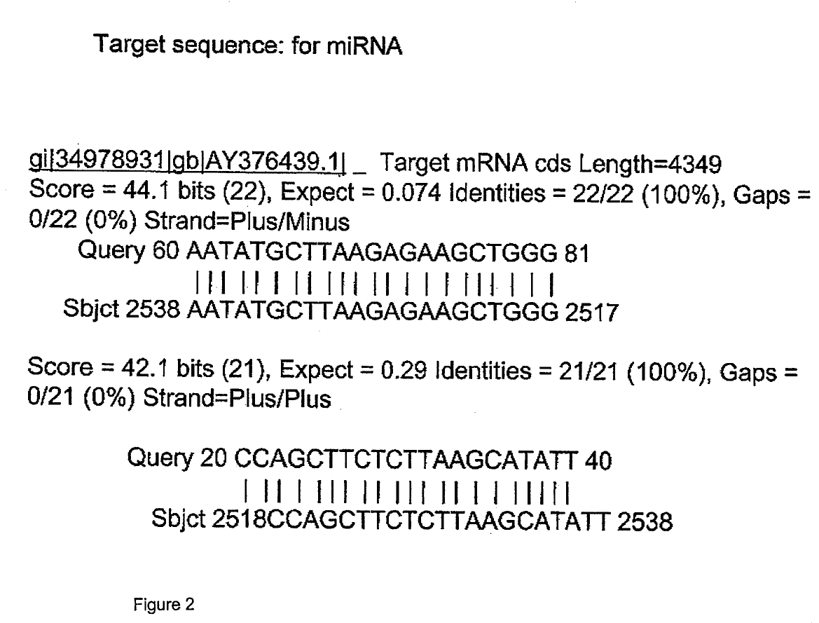

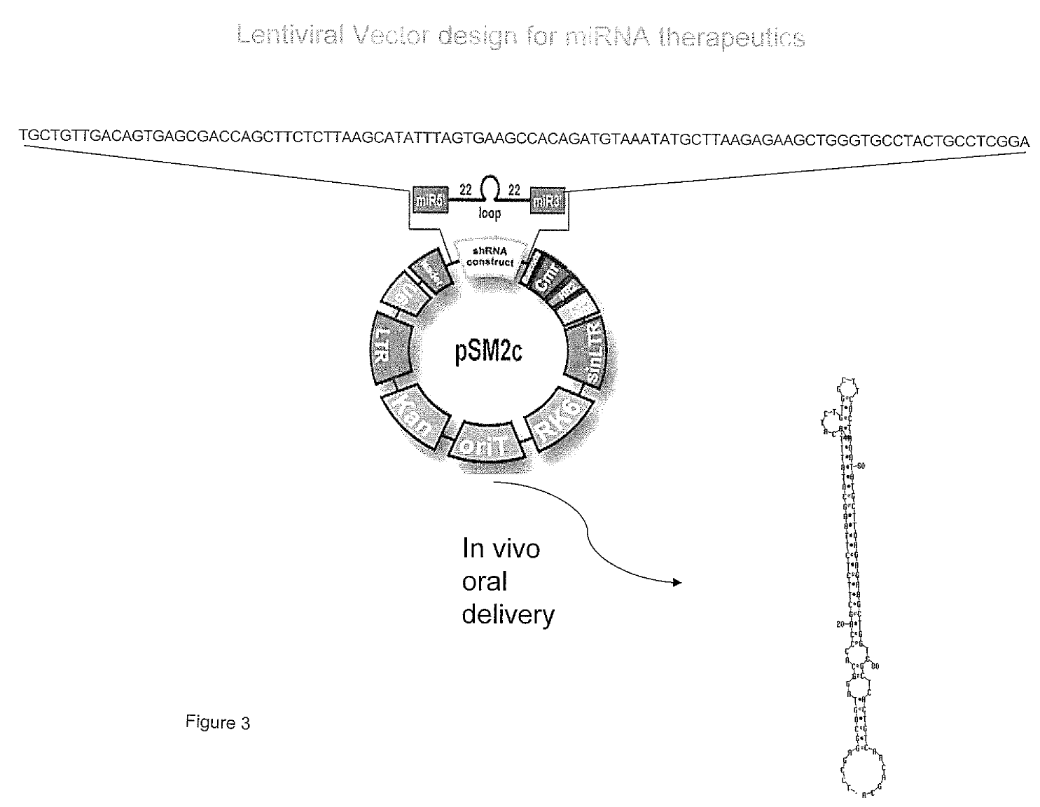

Referring to FIGS. 2 and 3, in one aspect of the invention, there is provided a micro-RNA comprising of or consisting of or consisting essentially of:

(SEQ ID NO. 1)

TGCTGTTGAC AGTGAGCGAC CAGCTTCTCT TAAGCATATT
TAGTGAAGCC ACAGATGTAA ATATGCTTAA GAGAAGCTGG
GTGCCTACTG CCTCGGA.

Preferably, the cancer is a head and neck cancer, which are characterized by the development of neoplastic lesions in the squamous epithelial compartment of the mouth cavity, lip, and pharynx.

As will be appreciated by one of skill in the art, once the construct is inside a suitable cell, the hairpin synthetic miRNA is transcribed and regulates ECT2 gene expression, thereby blocking crosstalk between NFKκβ and MAP kinase inflammatory pathways.

In some embodiments, treatment of an individual in need of such treatment with the recombinant construct ameliorates constitutive inflammatory conditions driving disease pathogenesis in epithelial, fibroblast and muscular components of human tissues and related pathological states, including but not exclusive to cancer development as discussed above.

In some embodiments, the construct is formulated in an orally active nanoparticulate gel for delivery of the construct to targetted tissues.

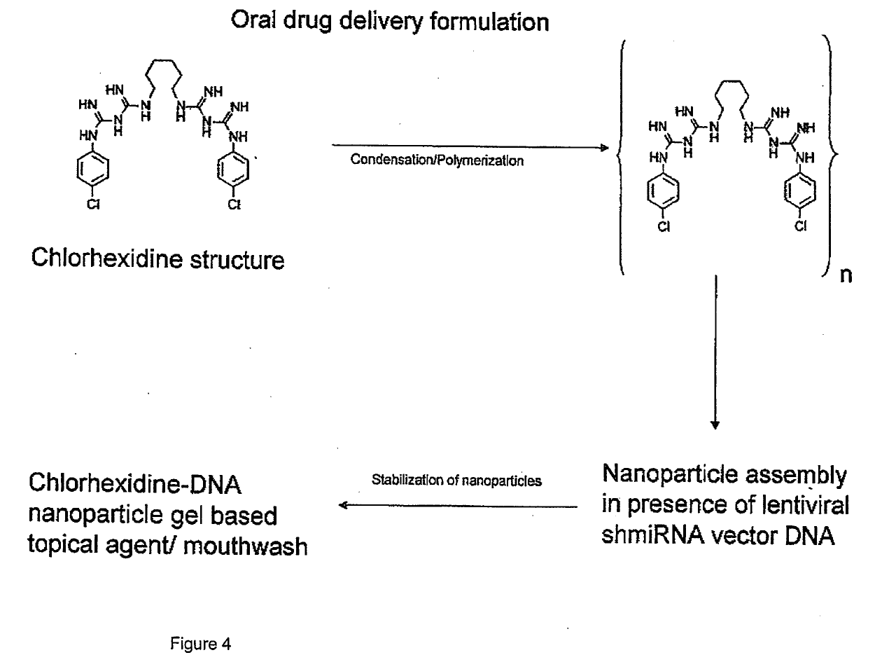

For example, as shown in FIG. 4, in one embodiment, an oral drug delivery formulation is prepared by condensation and polymerization of a chlorhexidine structure which is then assembled into nanoparticles in the presence of the shmiRNA vector shown in FIG. 3 and discussed above. As will be appreciated by one of skill in the art, the chlorhexidine-vector nanoparticle mixture can then be incorporated into a gel-based topical agent for application to the tissues of the mouth as discussed herein.

In other embodiments, ect2 activity may be modulated using double-stranded RNA species mediating RNA interference (RNAi) or natural and synthetic small molecule inhibitors of ECT2 gene transcription activity. RNAi is the process of sequence-specific, post-transcriptional gene silencing in animals and plants, initiated by double-stranded RNA (dsRNA) that is homologous in sequence to the silenced gene. Methods relating to the use of RNAi to silence genes in C. elegans, Drosophila, plants, and mammals are known in the art (Fire A, et al., 1998 Nature 391:806-811; Fire, A. Trends Genet. 15, 358-363 (1999); Sharp, P. A. RNA interference 2001. Genes Dev. 15, 485-490 (2001); Hammond, S. M., et al., Nature Rev. Genet. 2, 110-1119 (2001); Tuschl, T. Chem. Biochem. 2, 239-245 (2001); Hamilton, A. et al., Science 286, 950-952 (1999); Hammond, S. M., et al., Nature 404, 293-296 (2000); Zamore, P. D., et al., Cell 101, 25-33 (2000); Bernstein, E., et al. Nature 409, 363-366 (2001); Elbashir, S. M., et al., Genes Dev. 15, 188-200 (2001); WO0129058; WO9932619, and Elbashir S M, et al., 2001 Nature 411:494-498) and one of skill in the art will understand how such constructs may be constructed using the ECT2 sequences as exemplified in SEQ ID No. 1.

Accordingly, in another aspect of the invention, there is provided an inhibitory nucleic acid molecule that decreases ect2 expression in a cell. The nucleic acid molecule may be single-stranded or double-stranded, for example, an siRNA or an shRNA or similar molecule. For example, the inhibitory nucleic acid molecule may be an antisense nucleic acid molecule such as a small interfering RNA or a double-stranded RNA that inhibits expression of native ECT2 transcripts, thereby inhibiting expression of ect2 peptides. For example, the inhibitory nucleic acid molecule may correspond to or be complementary to at least a fragment of an Ect2 nucleic acid molecule. It is to be understood that said nucleic acid molecule will correspond to or be complementary to a sufficiently large fragment of an ECT2 nucleic acid molecule such that the inhibition or interference is specific to ECT2/ect2. Furthermore, the inhibitory nucleic acid molecule that targets Ect2 may be complementary or correspond to two or more non-contiguous sequence portions of an ECT2 nucleic acid molecule. For example, dsRNAs are typically 21 or 22 base pairs, but may be shorter or longer. It is of note that as discussed herein such inhibitory nucleic acid molecules are well known in the art as are modifications which can be made to such molecules to alter specific properties of the molecules, for example, to improve or limit resistance to degradation, half-life and the like. It is also important to note that such inhibitory nucleic acid molecules do not necessarily have to be perfectly complimentary to an ECT2 sequence as 1, 2, 3, 4, 5 or more mismatches may be tolerated. An exemplary ECT2 nucleic acid sequence may be found in SEQ ID No. 2 although as will be appreciated by those of skill in the art, the ECT2 sequence is known and may be determined from a variety of sources, for example, GenBank Accession NC_{—}000003.10 GI89161205.

The inhibitory nucleic acid molecule may be delivered to the site to be treated by a variety of means, for example but by no means limited to in a liposome, a polymer, a microsphere or a vector.

In some embodiments, there is provided a vector comprising an inhibitory nucleic acid molecule that decreases ect2 peptide expression in a cell as discussed herein. Suitable vectors include but are by no means limited to retroviral, adenoviral, adeno-associated viral and lentiviral vectors as well as other suitable vectors known in the art. Preferably, the vector includes a promoter suitable for expression in a mammalian cell operably linked to the inhibitory nucleic acid molecule.

Also provided are methods for modulation of ect2 protein translation activity. As will be apparent to one of skill in the art, these methods may be used to regulate inflammation triggered in epithelial components of biological tissues and dependent disease pathogenesis. For example, primary assays can be used to identify or confirm a modulator's specific biochemical or molecular effect with respect to the ECT2 nucleic acid or ect2 protein. In general, secondary assays further assess the activity of an ECT2-modulating agent identified by a primary assay and may confirm that the modulating agent affects ect2 in a manner relevant to the NFkB and MAP Kinase signaling pathways.

For small molecule modulators, screening assays are used to identify candidate modulators. Screening assays may be cell-based or may use a cell-free system that recreates or retains the relevant biochemical reaction of the target protein (reviewed in Sittampalam G S et al., Curr Opin Chem Biol (1997) 1:384-91 and accompanying references). As used herein the term “cell-based” refers to assays using live cells, dead cells, or a particular cellular fraction, such as a membrane, endoplasmic reticulum, or mitochondrial fraction. Cell-based screening assays usually require systems for recombinant expression of ect2 and any auxiliary proteins demanded by the particular assay. The term “cell free” encompasses assays using substantially purified protein (either endogenous or produced using recombinant DNA methods), partially purified cellular extracts, or crude cellular extracts. Screening assays may detect a variety of molecular events, including protein-DNA interactions, protein-protein interactions (e.g., receptor-ligand binding), transcriptional activity (e.g., using a reporter gene), enzymatic activity (e.g., via a property of the substrate), activity of second messengers, immunogenicty and changes in cellular morphology or other cellular characteristics. Appropriate screening assays may use a wide range of detection methods including fluorescent (Klebe C, et al., Biochemistry (1995) 34:12543-12552), radioactive (Hart M, et al., Nature (1991) 354:311-314), calorimetric, spectrophotometric, and amperometric methods, to provide a read-out for the particular molecular event detected, often in high throughput screening (HTS) formats (for example, see Hertzberg R P, and Pope A J, Current Opinion in Chemical Biology (2000) 4:445-451).

Assays for binding agents include screens for compounds that modulate ect2 interaction with a natural ect2 binding target. The ect2 polypeptide used in such assays may be fused to another polypeptide such as a peptide tag for detection or anchoring, etc. In a particular embodiment, the binding target is RhoA, RhoC, Rac, or Cdc42, or portion thereof that provides binding affinity and avidity to the subject Ect2 polypeptide conveniently measurable in the assay and preferably comparable to the intact RhoA, RhoC, Rac, or Cdc42. The ect2 and binding target are incubated in the presence and absence (i.e. control) of a candidate ect2 modulating agent under conditions whereby, but for the presence of the candidate modulating agent, the ect2 polypeptide specifically binds the cellular binding target, portion or analog with a reference binding affinity. After incubation, the agent-biased binding between the ect2 polypeptide and one or more binding targets is detected by any of a variety of methods depending on the nature of the product and other assay components, such as through optical or electron density, radioactive emissions, non-radioactive energy transfers, etc. or indirect detection with antibody conjugates, etc. A difference in the binding affinity of ect2 to the target in the absence of the agent, as compared with the binding affinity in the presence of the agent indicates that the agent modulates the binding of the ect2 to the ect2 binding target. A difference, as used herein, is statistically significant and preferably represents at least a 50%, preferably at least 60%, more preferably 75%, and most preferably a 90% difference between the two values, for example, an agent which increases ect2 binding to the target may have at least 50% greater binding compared to an untreated control.

We developed a solid-phase chemiluminiscent high throughput assay format to measure activity of ect2, and other GEFs. The GTPase/GEF activity is evaluated by measuring the binding of the activating ligand -GTP in solid phase. In this assay, the GTPase (such as Rho or Rac) is adsorbed to the bottom of commercially available plates, such as Flashplate (Perkin Elmer Life Sciences), which have scintillant coated on the bottom and sides of the wells. The plates are then washed to remove excess protein. A test compound (candidate modulating agent) is added, followed by GEF (such as ect2, or a functional ect2 fragment such as a fragment comprising the Dbl homology domain), followed by ³⁵S labelled GTP. When the radioisotope is associated with the solid phase, it is measured in a scintillation counter just as if liquid scintillant had been added. Thus, following incubation, the plates are simply counted without further processing, since only ³⁵S-GTP that is exchanged onto the GTPase will be detected. Unbound radioactive GTP remains in solution and is undetectable. Magnesium chloride is used as a negative control. In the absence of GEF, 2 mM MgCl₂ prevents GTP from binding, and thus reduces the number of cpm/well. Inclusion of GEF in the assay will rescue the MgCl₂ inhibited exchange.

Other preferred assay formats use fluorescence technologies, including fluorescence polarization, time-resolved fluorescence, and fluorescence resonance energy transfer. These systems offer means to monitor protein-protein or DNA-protein interactions in which the intensity of the signal emitted from dye-labeled molecules depends upon their interactions with partner molecules (e.g., Selvin P R, Nat Struct Biol (2000) 7:730-4; Fernandes P B, Curr Opin Chem Biol (1998) 2:597-603; Hertzberg R P and Pope A J, Curr Opin Chem Biol (2000) 4:445-451). We developed an FMAT (Fluorescent Microvolume Assay Technology) assay format to measure the protein-protein interaction of a GEF and GTPase, whereby GST-fused GTPase (such as RhoA, RhoC, or Rac) is attached to polystyrene beads and the GEF (such as Ect2) is labeled with Cy5 (a long wavelength fluorophore, available from Amersham). When the GTPase and the GEF are associated, there is an increase in fluorescence associated with GTPase beads, which settle to the bottom of the well and are detected using an FMAT 8100 HTS system (Applied Biosystems). Potential inhibitors interfere with the GEF-GTPase association with subsequent decrease in fluorescence.

For antibody modulators, appropriate primary assays test the antibody's specificity for and affinity to the ect2 protein. Methods for testing antibody specificity and affinity are well known in the art. Alternatively or additionally, primary assays for antibody modulators may comprise the screening assays described above, used to detect the ect2 modulator's specific activity.

For nucleic acid modulators, primary assays may test the ability of the nucleic acid modulator to inhibit ECT2 mRNA or protein expression. In general, expression analysis comprises comparing ECT2 expression in like populations of cells (e.g., two pools of cells that endogenously or recombinantly express ect2) in the presence and absence of the nucleic acid modulator. Methods for analyzing mRNA and protein expression are well known in the art. For instance, Northern blotting, slot blotting, ribonuclease protection, quantitative RT-PCR (e.g., using the TaqMan.RTM., PE Applied Biosystems), or microarray analysis may be used to confirm that ECT2 mRNA expression is reduced in cells treated with the nucleic acid modulator (e.g., Current Protocols in Molecular Biology (1994) Ausubel F M et al., eds., John Wiley & Sons, Inc., chapter 4; Freeman W M et al., Biotechniques (1999) 26:112-125; Kallioniemi O P, Ann Med 2001, 33:142-147; Blohm D H and Guiseppi-Elie, A Curr Opin Biotechnol 2001, 12:41-47). Proteins are most commonly detected with specific antibodies or antisera directed against either the ect2 protein or specific peptides. Protein expression can be monitored using by a variety of means including Western blotting, the enzyme-linked immunosorbent assay (ELISA), or in situ detection (Harlow E and Lane D (eds.) Using Antibodies: A Laboratory Manual, 1999, Cold Spring Harbor Laboratory Press, New York).

### Secondary Assays

Secondary validation can use essentially the same assays used to functionally validate the participation of an ECT2 gene in the NFkB and MAP kinase pathway. Whereas the afore-described functional validation assays generally compare cells expressing altered levels of an ect2 protein, secondary validation assays generally compare like populations of cells (e.g., two pools of wild type cells) in the presence and absence of the candidate modulator.

In another embodiment, secondary validation may use the same assays used for high throughput screening. These methods can confirm the activity of a modulator not identified through high throughput screening, such as an antibody or an antisense oligonucleotide modulator, or can confirm the activity of a small molecule modulator identified using a different high throughput screening assay. These assays may also be used to confirm the specificity of a candidate modulator.

Additionally, the modulator is assayed for its effectiveness on the ect2 in the NFκB transcription activity related manner. Such assays include cell cycle, apoptosis, proliferation, angiogenesis and anti-hypoxia induction assays, among others, as described above. To assess the role of modulators, these assays are performed in presence or absence of the modulator in NFKB and MAP Kinase activity normal and mutated backgrounds of cell lines (ATCC) or animal models (Jackson laboratories) of disease.

### Therapeutic and Diagnostic Applications

When used for anti-cancer prevention therapy in a patient, ECT2 expression regulating agents are administered to the patient in therapeutically effective amounts that eliminate or reduce the patient's tumor burden. They will normally be administered parenterally, when possible at the target cell site, or intravenously. The dose and dosage regimen will depend upon the nature of the cancer (primary or metastatic), its population, the target site, the characteristics of the particular immunotoxin (when used), e.g., its therapeutic index, whether the agent is administered in combination with other therapeutic agents, and the patient's history. Antibodies that specifically bind ect2 may be used for the diagnosis of conditions or diseases characterized by expression of ECT2, or in assays to monitor patients being treated with Ect2 modulating agents. Diagnostic assays for ect2 include methods which utilize the antibody and a label to detect ect2 in human body fluids or extracts of cells or tissues. The antibodies may be used with or without modification, and may be labeled by joining them, either covalently or non-covalently, with a reporter molecule.

Diagnosis of conditions characterized by expression of ECT2 may also be accomplished by any of a variety of methods such as Northern or TaqMan.RT-PCR. The following experimental section and examples are offered by way of illustration and not by way of limitation.

### EXAMPLES

### I. High Throughput Fluorescent or Chemilumiscent or Radioactive Homogeneous Assay

Various combinations of fluorescently (with N-Methylanthraniloyl, Bodipy or other commonly used fluorophores) or chemiluminiscent substrates labeled GTP, GDP, dGTP, or dGTP and ect2 are added to each well of a 96-well plate, along with a test compound of choice. Fluorescent measurements (of over 500 nm to reduce background fluorescence) or radioactivity measurements indicative of the exchange reaction are then taken.

The above assay may be performed where all components are in solution, or alternatively, where at least one component is attached to beads that are 10 nm or larger in diameter (such as SPA beads from Amersham, Alpha screen beads from Packard, or FMAT beads from PE Biosystems).

### II. High Throughput ELISA Format Assay

Various combinations of Glutathione-S-transferase/RhoA, Rhoc, RAC, or CDC42 polypeptide fusion protein and biotinylated Ect2 are added to each well of a microtiter plate (Reacti-Bind Streptavidin-Coated, White Polystyrene Plates (#15118B), which have been blocked by Super-Blocking Reagent from Pierce) in assay buffer (0.01M HEPES, 0.15M NaCl, 0.002M MgCl₂). Test compounds are then added to each well, and incubated at room temperature for 1 hour. Anti-GST, rabbit and anti-rabbit antibodies are then added to each well and incubated on ice for 1 hour. Plates are then washed with water, diluted Supersignal substrate is added to each well, and chemiluminescence is then measured.

III. Solid Phase Rac1-Ect2 screen
3×30 plates/day
Day 1
Reconstitute 4×10 mg GST-Rac1 in 4×10 ml Assay Buffer
Prepare 3 L Assay Buffer (to 1 L 1.4 mM Tris pH7.5, 5 mM MgCl₂, 0.3% sucrose, 0.1% dextran add 1 ml 1M DTT/L)
Dilute GST-Rac1 into 100 ml Assay Buffer
Mix
Dilute into 1 L Assay Buffer
Mix
Dilute into 2 L Assay Buffer.
Giving a final volume of 2 L of 10 ug/ml GST-Rac1 in Assay Buffer.
Coat 90 Flashplates (Perkin Elmer Life Sciences) with 0.5 ug/well GST-Rac1 (50 ul of 10 ug/ml GST-cdc42 in Assay Buffer)
Place at 4° C. overnight
Prepare 2 L TBS (50 mM Tris-HCl, pH 7.4, 150 mM NaCl).

### Day 2.

Thaw 1 vial (1 mCi)[³⁵S]GTPγS.
Wash 30 GST-cdc42 coated plates 3×70 ul TBS
Dilute compound in plates by addition of 10 ul Assay Buffer
Transfer 5 ul compound dilution to assay plates
Prepare 0.1 L of assay buffer containing 1 flourescent or chemiluminiscent tagged GTP substrate, 500 nM ect2
Add 5 ul/well ect2/GTP (columns 1&2 receive buffer alone)
Seal
Incubate@room temp×1 hour
Count in the Trilux Scintillation counter
Prepare the remaining 2×30 plates as described above and store at room temp.

### IV. FMAT GEF Assay

0.5 ml Protein G polystyrene beads (7 u, 0.5% w/v Spherotech [Libertyville, III.]) are washed three times with PBS and resuspended in 0.5 ml PBS. For monitoring of biomolecular binding events, Anti-GST (0.25 ug BIAcore [Uppsala, Sweden]) is added and incubated at room temperature for 30 minutes. The beads are then washed three times with PBS and resuspended in 0.5 ml PBS. The sample is split into 2×0.25 ml aliquots and 2.5 ug of either GST or GST-RhoA is added and incubated at room temperature for 30 minutes. The beads are then washed three times with PBS and resuspended in 0.25 ml PBS.

(His)6 tagged Ect2-dbl domain is labeled with Cy 5 using a Cy5 monoclonal antibody labeling kit according to the manufacturers instruction (Amersham).

To 400 ul of PBS add 4 ul of either “RhoA-beads” or “GST-beads” giving a final concentration of 20 nM RhoA or GST. Add 200 nM Cy5-Ect2_dbl. Mix and aliquot 8×50 ul into a 96 well FMAT plate. Incubate at room temperature for 1 hour and read in the Cy5 detecting channel of an FMAT 8100 HTS system.

While the preferred embodiments of the invention have been described above, it will be recognized and understood that various modifications may be made therein, and the appended claims are intended to cover all such modifications which may fall within the spirit and scope of the invention.

### REFERENCES

Saito S et al., J. Biol. Chem. (2004) 279, (8):7169-7179,

Niiya F et al., Oncogene (2006): 25827-837,

Scoumanne and Chen, Cancer Research (2006)66 (12): 6271.

Lehner, C. F., J. Cell Sci., 1992, 103:1021-1030,

Adams, M. D., “EST185199 Colon carcinoma (HCC) cell line Homo sapiens cDNA 5′ end similar to similar to transforming protein, mRNA sequence” Genbank GI No.1965630, Apr. 19, 1997.

Hedge, P., et al., “EST377993 MAGE resequences, MAGI Homo sapiens cDNA, mRNA sequence” Genbank GI No. 8155756, Jun. 1, 2000.

NCI-CGAP, “tu89e03.×1 NClaCGAPaGas4 Homo sapiens cDNA clone IMAGE:2258236 3′ similar to TR:Q07139 Q07139 ECT2 Oncogene” Genbank GI No. 5636530, Dec. 15, 1999.

NIH-MGC, “UI-HF-BN0-aln-c-10-0OUI.r1 NIHaMGCa50 Homo sapiens cDNA clone IMAGE:3080082 5” Genbank GI No. 7142453, Mar. 2, 2000.

Dias Neto, E., “QV1-BT0631-150200-071-f05 BT0631 Homo sapiens cDNA, mRNA sequence” Genbank GI No. 8471000, Jun. 12, 2000.

NIH-MGC, “UI-HF-BN0-ala-h-11-0-UI.r1 NIHaMGCa50 Homo sapiens cDNA clone IMAGE:3079149 5” Genbank GI No. 7142100, Mar. 2, 2000.

Dias Neto, E., “QV1-BT0631-280200-084-d11 BT0631 Homo sapiens cDNA” Genbank GI No. 8471150, Jun. 12, 2000.

Hegde P., “EST382885 MAGE resequences, MAGK Homo sapiens cDNA” Genbank GI No. 8160647, Jun. 1, 2000.

NCI-CGAP, “zs92g10.r1 NClaCGAPaGCB1 Homo sapiens cDNA clone IMAGE:7040994 5′ similar to TR:G293332 G293332 ECT2 Protein. mRNA sequence” Genbank GI No.1921407, Aug. 15, 1997.

Hillier, L., “zq51a07.r1 Stratagene neuroepithelium (#937231) Homo sapiens cDNA clone IMAGE:645108 5′ similar to TR:G293332 G293332 ECT2 Protein” Genbank GI No. 1801929, Jan. 27, 1997.

Miki, T., “Mouse oncogene (ect2) mRNA, complete cds” Genbank GI No. 293331, Jun. 12, 1999.

Miki, T., “ect” Genbank GI No. 293332, Jun. 12, 1993.

Miki, T., “Mouse oncogene (ect2) mRNA, complete cds” Genbank GI No. 293331, Jun. 12, 1993.

Tatsumoto et al, Human ECT2 is an exchange factor for Rho GTPases, phosphorylated in G2/M phases, and involved in cytokinesis. J. Cell Biol. 147:921-927, 1999.

1. An inhibitory nucleic acid molecule that corresponds to or is complementary to at least a fragment of an Ect2 nucleic acid molecule and that decreases Ect2 expression in a cell.

2. A micro-RNA comprising a nucleotide sequence as set forth in SEQ ID No. 1.

3. A vector comprising a micro-RNA comprising a nucleotide sequence as set forth in SEQ ID No. 1 operably linked to a suitable promoter.

4. The vector according to claim 3 wherein the promoter is capable of expression of any shmiRNA recombinant construct in human cells.

5. A pharmaceutical composition comprising the vector of claim 3 and a nanoparticle delivery matrix.

6. The composition according to claim 5 wherein the composition is arranged for oral and other epithelial tissue administration as a topical agent.

7. A method of preventing cancer in an individual having a precancerous lesion of epithelial origin comprising administering to said individual an effective amount of an inhibitory nucleic acid molecule that corresponds to or is complementary to at least a fragment of an Ect2 nucleic acid molecule and that decreases Ect2 expression in a cell.


SEQUENCE LISTING (2 sequences)

SEQ ID NO: 1 - DNA, 97 bp, artificial
  microRNA sequence
tgctgttgacagtgagcgaccagcttctcttaagcatatttagtgaagccacagatgtaaatatgcttaagagaagctgggtgcctactgcctcgga

SEQ ID NO: 2 - DNA, 1262 bp, human
gttgttcactttaagacatctattcagttttcatcttaactagatttgagttgtagtactatgtgtgttccaccaatgtggaaactgaggtttaactgactatgtgcagggctgagacaggaagctgagttgcctgattttgaagtgtcacataacagcatacagtttaaactgttactttgctaagactctggaggaaggtatttgtatattttattaatctgaagaagtaggaaaggggaaagtctgacatgtagctctcgttaaaggtgggtcctctgtatatttgcctaatatttagaaatttactctcagtaaagttgacttattgaaaattgataaaaaaatctctaaaaaaatttttttagagagacagggtcttgctctgtcacctagactgactggagtgcagtggtgggatcacagctcactacaacctcaaactcctggattcaagtgattcgcccacctcagcctcttgagtagctactataggcacctgccacccacacctggctaatttttaaattttttgtagagatggggtcttgctatgttgcccagtctggtgttgaagtcctgccctcaagtgatcctcctgccttggcctcccaaagtgctgggatttcagatgtgagcccccaacccaccctgatgaaaacttgttaaaccttttattccctgtctagcttttaaactcttcttaagtcctggaaaacttaaaaaaggattttctttcatagtccagagttatattggcactcacccatgcataattgttttgtcaaaccttttaggggagaggtgtttttaaagaattatgaaaatactgacaagggaccagcccaattataccggagaggtttagagacatccacagaggatgagtcaagagtgttggattgggggttggaagacaagataggtggagagtcgagcactaaatgcaaatagatgtgatgtaaattatgatttgtgcctgaaaaatagctgagaaattcattgttgctgttgcatagattgtatagggactggggtagttacagatggtgagggtgaatagttaggcggggaccaaattgtgaacttggtggtgaatttggacttggacatataaggagtactcatggaagttgttaagcagggaagtgatatggtcacatttgtatttaggtgcatattcctgaaaaccatgtggagagtgatttctaaggaaactgatggtaaagttgtgtggagctggtttattagaccaagtgacc